(19) United States
(12) Patent Application Publication
(10) Pub. No.: US 2024/0263215 A1
(43) Pub. Date: Aug. 8, 2024

(54) ASSAYS

(71) Applicant: QBIOTIX LIMITED, Ledbury (GB)

(72) Inventors: Ian Peter Thrippleton, Ledbury (GB); Tyler Howell Bray, Ledbury (GB)

(73) Assignee: QBIOTIX LIMITED, Ledbury (GB)

(21) Appl. No.: 18/564,698

(22) PCT Filed: May 27, 2022
(86) PCT No.: PCT/GB2022/051372

§ 371 Date: Nov. 28, 2023

(30) Foreign Application Priority Data
May 28, 2021 (GB) 2107664.1

(51) Int. Cl. C12Q 1/6818; G01N 21/64

(52) U.S. Cl. CPC C12Q 1/6818; G01N 21/6486

(57) ABSTRACT

The present invention relates to a nucleic acid probe for use in the detection of a functional splicing protein. The invention also relates to a method for the detection of a functional splicing protein, and a kit for the same. The invention further provides methods to quantify the presence of the level of functional protein in the sample.

### RELATED APPLICATIONS

This application claims priority to and benefits of GB Patent Application number 2107664.1 filed 28 May 2021, entitled ‘Assays’.

### FIELD OF THE INVENTION

The present invention relates to novel probes and assays formats for the detection of splicing proteins in a biological sample, and kits and methods for detecting said proteins.

### SEQUENCE LISTING

The instant application contains a Sequence Listing which has been filed electronically in ASCII format and is hereby incorporated by reference in its entirety. Said ASCII copy, created on 25 May 2022, is named Qbiotix_Split_ST25.txt and is 2 KB in size.

### BACKGROUND TO THE INVENTION

Out of the approximately 240 human proteins that are known to harbour pathogenic intrinsically disordered regions (IDRs), approximately 70 of them are DNA/RNA binding proteins containing RNA recognition motifs (RRM). Those implicated in the pathogenesis of Neurodegenerative Diseases (NDs) include TDP-43, FUS, TAF 15, and EWRS1 (Prasad et al 2019 Molecular Mechanisms of TDP-43 Misfolding and Pathology in Amyotrophic Lateral Sclerosis. Front. Mol. Neurosci. 12: doi.org/10.3389/fnmol.2019.00025) which are heterogenous nuclear ribonucleoproteins (hnRNP). The changes that occur in this role as part of disease progression are known to be significant with alternative splicing leading to the up and downregulation of splicing events, each change specific to the protein in question.

Splicing is conducted by the spliceosome, a large RNA-protein complex composed of five small nuclear ribonucleoproteins (snRNPs). Assembly and activity of the spliceosome occurs during transcription of the pre-mRNA. The RNA components of snRNPs interact with the intron and are involved in catalysis. In many cases, the splicing process can create a range of unique proteins by varying the exon composition of the same mRNA. This phenomenon is then called alternative splicing. Alternative splicing can occur in many ways. Exons can be extended or skipped, or introns can be retained. It is estimated that 95% of transcripts from multiexon genes undergo alternative splicing, some instances of which occur in a tissue-specific manner and/or under specific cellular conditions.

The five snRNPs involved in splicing are U1, U2, U5, U4, and U6, these small proteins are known to be required for splicing and are some of the most abundant proteins within cells. Together with other larger splicing proteins (such as the hnRNP family) the snRNPs form the spliceosome. It is however noteworthy that understanding of the spliceosome and its behaviours is still in its infancy. Collectively it is understood that the spliceosome acts on pre-mRNA removing introns and connecting exon segments for subsequent protein translation.

Protein and DNA/RNA recognise each other by ‘induced fit’ (herein after “bind”) causing conformational changes in either partner or both. The folding of the protein including important secondary structures such as B-sheets and a-helixes, as well as their surrounding loop structures are crucial for nucleic acid recognition.

For some time understanding of splicing mechanisms has been stunted by an absence of information in the literature regarding the exact structural mechanisms employed by the nucleic acid binding/splicing proteins, including how they recognize and discriminate between their DNA and RNA targets.

However, Kashap et al. (2015) analysis of the TAF-15-RRM (a member of the FET family implicated in mRNA splicing) has provided insight into the dynamics of this process (Kashap et al. (2015) Structural delineation of stem-loop RNA binding by human TAF15 protein (Scientific Reports, 5:17298.). Elucidating that rather than classic stacking interactions that occur across nitrogen bases, moderate-affinity hydrogen bonding networks between the nitrogenous bases in the stem-loop of the RNA and the face of the RRM surface mediate this interaction. This group's conclusion is that DNA/RNA binding is dependent on the structural elements of the RNA rather than simply sequence alone.

The transactive response (TAR) DNA/RNA binding protein TDP-43 is part of the hnRNP protein family with a well-established and significant role in alternative splicing (1). TDP-43 is of interest due to the established role of its aggregates in many neurodegenerative disorders (ND), namely Amyotrophic Lateral Sclerosis (ALS), frontotemporal lobar degeneration (FTLD), Alzheimer's disease (AD) and related tauopathies (Bhardwaj et al. 2012 Characterising TDP-43 interaction with its RNA targets. Nucleic Acids Res. 41 (9): 5062-5074).

The TDP-43 protein consists of four regions, the N-terminal, RRM1, RRM2 and the C-terminal. Although some genetic mutations in the TARDBP gene that encodes for TDP-43 have been associated with disease phenotype, these mutations are limited in number and generally appear within the low complexity, less highly conserved C-terminal region (a common feature of proteins that misfold). Notably, more than 90% of the ND cases mentioned are sporadic, denoting that the patients do not carry any genetic mutations, firmly placing TDP-43's role in pathology within the remit of protein only disease.

The two RNA recognition motifs (RRM) of TDP-43 are known to play an active role in binding and alternative splicing of RNA and DNA, with RRM1 posited as the main binding site, assisted by RRM2 (Buratti E, Baralle F E. Characterization and functional implications of the RNA binding properties of nuclear factor TDP-43, a novel splicing regulator of CFTR exon 9. J Biol Chem. 2001 Sep. 28; 276(39):36337-43. doi: 10.1074/jbc.M104236200. Epub 2001 Jul. 24. PMID: 11470789.). It is reported that UU/UG/GU rich repeats bind with high affinity to RRM1, even when interspersed by other nucleotides. To facilitate binding of DNA/RNA to TDP-43 the maintenance of accessible (not currently bound) dinucleotide repeats is required, these cannot have complex secondary and tertiary structure, and must represent a significant local increase in concentration of dinucleotide repeats more than six dinucleotide repeats). This enables the association and dissociation constants of the protein to allow for binding to occur, splicing to take place, diffusion of the spliced sequence and hence for the process to repeat (Bhardwaj, Amit et al. “Characterizing TDP-43 interaction with its RNA targets.” Nucleic acids research vol. 41, 9 (2013): 5062-74. doi:10.1093/nar/gkt189).

TDP-43 and other splicing proteins are well established as a biomarker for protein pathology and disease progression; however, these assay procedures are often invasive and time consuming. For example, Scialo et al. (2020) demonstrated that there are significant detectable differences the templating response of TDP-43 between ALS and FTLD patients, using real time quaking induced conversion rate (Scialo et al. 2020 TDP-43 real-time quaking induced conversion reaction optimization and detection of seeding activity in CSF of amyotrophic lateral sclerosis and frontotemporal dementia patients. Brain. Comms. 2: doi.org/10.1093/braincomms/fcaa142). French et al. (2019) studied the oligomerisation of TDP-43 on its conversion to amyloid, identifying distinct oligomeric complexes that form prior to the formation of large aggregates. Interestingly this and other studies have found that the RRM1 is required for this process to occur and that significant interactions with favourable dinucleotide repeat regions strongly inhibit this oligomerization and aggregation (French et al. (2019) Detection of TAR DNA-binding protein 43 (TDP-43) oligomers as initial intermediate species during aggregate formation. J. Biol. Chem. 294: 6696-6709). This suggests that the misfolding process of TDP-43 significantly alters the structural and kinetic parameters of its interactions with nucleic acid sequences, potentially making other substrates more favourable or alternatively changing the level of interaction, via either up or down regulation. Supported by Xiao et al. (2011) study which demonstrated significant deregulation of RNA targets of TDP-43 in ALS patients (Xiao et al. 2011 RNA targets of TDP-43 identified by UV-CLIP are deregulated in ALS. Mol. Cell. Neurosci. 47: 167-180). De Schaepdryver et al. (2019) found significant differences in protein blood markers are present in ALS patients in excess of 18 months before diagnosis, and even prior to symptom development (De Schaepdryver et al. (2019) Serum neurofilament heavy chains as early marker of motor neuron degeneration. Ann. Clin. Transl. Neurol. 6: 1971-1979).

FUS (Fused-in-Sarcoma) is a DNA/RNA binding protein known to play a role in various cellular processes including DNA/RNA splicing and autoregulation of its own expression (Yamaguchi and Takanashi (2016) FUS interacts with nuclear matrix-associated protein SAFB1 as well as Matrin3 to regulate splicing and ligand-mediated transcription. Scientific Reports, 6: 35195). Similarly, to TDP-43 it has been implicated in the development and progression of ALS and FTLD, with findings suggesting even slight changes in the levels of splicing compromise FUS autoregulation and induce pathogenicity, suggesting that this is a tightly regulated process and an important protein to monitor for early disease progression (Zhou et al. (2013) ALS-Associated FUS mutations result in compromised FUS alternative splicing and autoregulation. PLOS GENETICS, 9(10): e1003895). With a role in nucleo-cytoplasmic shuttling this protein is thought to bind to pre-mRNA in the nucleus, translocating the sequence to interact with the snRNPs (Yamaguchi and Takanashi, 2016 FUS interacts with nuclear matrix-associated protein SAFB1 as well as Matrin3 to regulate splicing and ligand-mediated transcription, Scientific Reports, 6; 35195). Although this suggests a snRNP dependency that is not clear with TDP-43, these proteins collectively form an important part of the spliceosome, playing crucial roles in the regulation and processing of DNA and RNA. The FUS binding motifs are targeted and specific and using these motifs, via the exact same process as employed with TDP-43 (simply switching the binding motifs on our probes) it should be possible to monitor the activity and quantify FUS.

The first published DNA/RNA binding motif for FUS was GGUG, subsequently CUGG, UGGU, GCUG, CCUG, UGGG and GUGG were identified (Takeda et al. (2017) Six GU-rich (6GUR) FUS-binding motifs detected by normalization of CLIP-seq by Nascent-seq. Gene, 618: 57-64). However, Wang et al. (2015) found that FUS bound with a significantly higher affinity to TERRA repeat regions, containing the repeat sequence UUAGGG (Wang et al. (2015) Nucleic acid-binding specificity of human FUS protein, Nucleic Acid Res. 43: 7535-7543).

Methods to detect such splicing proteins have traditionally relied on antibody-based assay or infer presence by the detection of nucleic acids.

Current diagnostic procedures to detect such splicing proteins are limited by their invasiveness, cost and the level of disease progression required to detect, due to absolute low levels of respective protein(s) present.

Approximately 50 human disorders relating to protein misfolding and aggregation are known to exist, these include Amyotrophic Lateral Sclerosis (ALS), frontotemporal lobar degeneration (FTLD), Alzheimer's disease (AD) and Parkinson's disease (PD) and its related tauopathies. Collectively known as the neurodegenerative disorders (ND), these protein pathologies are posited to place significant economic and emotional burden on the people, communities and governments of the world. Despite this there are currently no accepted treatments for the NDs, thus making them fatal incurable diseases.

By identifying high risk patients or those early on in disease progression is a critical step in assessing the effectiveness of treatment or drug trials (Hussian et al. 2018 Neurodegenerative Diseases: Regenerative Mechanisms and Novel Therapeutic Approaches. Brain Sci. 8: 10.3390/brainsci8090177). Nevertheless, currently the diagnosis and assessment of the neurodegenerative disorders (ND) is largely based on clinical presentation and assessment which often happens late on in the disease progression. However, given the propensity of these conditions to provide a variety of symptoms and the reliance of these methods on the accuracy of self-reporting, commonly disease progression is advanced when a diagnosis is confirmed, further narrowing effective treatment monitoring and the fruitfulness of drug trials. This is in sharp contrast to a variety of other conditions whereby biofluid quantitative biomarkers, form an integral part of diagnostics and treatment selection (Obrocki et al. 2020 Perspectives in fluid biomarkers in neurodegeneration from the 2019 biomarkers in neurodegenerative diseases course—a joint PHD student course at University College London and University of Gothenburg. Alzheimer's Res. & Therapy, 12: doi.org/10.1186/s13195-020-00586-6).

Efforts to understand and monitor the pathology of proteins involved in NDs has routinely focused on the C-terminal region due to it (usually) being the location of IDRs, its propensity for templating action, low complexity and aberrant mutations (French et al. 2019 Detection of TAR DNA-binding protein 43 (TDP-43) oligomers as initial intermediate species during aggregate formation. J. Biol. Chem. 294: 6696-6709). However, it is becoming clear that the functional role of some of the proteins involved in ND, has a far more central role to disease pathology than previously thought.

Thus, it is an objective of the invention to permit efficient monitoring of the functional roles of these splicing proteins.

### SUMMARY OF THE INVENTION

The present invention addresses some of the problems with the prior art methods. In particular the present invention provides an assay that permits the detection of splicing proteins, based on their functional splicing activity, in easily accessible biofluids, in a homogeneous assay without the need for lengthy wash and handling steps. The invention further provides methods to quantify the presence of the level of functional protein in the sample.

In a first aspect the present invention provides a nucleic acid probe comprising a binding motif for RNA-Protein recognition and use as a substrate in the detection of a functional splicing protein, the probe comprising:
a) a pseudo intron flanked by a first pseudo exon and a second pseudo exon; wherein the pseudo intron contains a 5′ splice site, a branch point and a 3′ splice site;
b) a repeat nucleotide motif as a recognition element for binding with a splicing protein; and
c) a signal generating component for generation of a signal upon excision of the pseudo intron and ligation of the first pseudo exon and the second pseudo exon.

The probe may be a deoxyribonucleic acid (DNA) or ribonucleic acid (RNA) probe. Wherein the probe is an RNA probe, it is preferred that all the 2′OH of the RNA sugar moiety of the RNA nucleotides are alkylated and typically methylated.

The recognition element comprises at least one recognition sequence for interaction with a splicing protein. Preferably, the recognition element comprises a first recognition sequence and a second recognition sequence for interaction with a splicing protein. Further recognition sequences may be provided. Typically, the first recognition sequence is located in the pseudo intron, and the second recognition sequence is located in either the first pseudo exon or the second pseudo exon. In an embodiment the first recognition sequence comprises all, or essentially all of the pseudo intron.

In an embodiment the probe of the invention comprises recognition sequences that bind to a splicing protein selected from the group of heterogeneous nuclear ribonucleoproteins (hnRNPs) such as the splicing protein TAR DNA-binding protein 43 (TDP-43) or Fused in Sarcoma (FUS). In other embodiments the probe binds to particular isoforms of such hnRNPs.

In an embodiment there is provided a probe wherein at least one of the recognition sequences binds to the RRM1 domain of TDP-43. Thus, in an embodiment the probe, the pseudo intron and the product formed in the presence of a functional splicing protein have the sequences as shown in table 1 and Table 2 below (SEQ ID No's 1 to 6).

TABLE 1
RNA Probe

| | | | |
|---|---|---|---|
| Probe | 5′ | Atto390- | 3′ |
| sequence | | uaaaaaaaacgaacacucccaaaaaaaaacaaaggcuucAUCUUUUUUUUUUUUA | |
| | | CUUUUUUUUUUUUCUUUGUuuuuuuuua-Atto495 (SEQ ID No. 1) | |
| | | Key: recognition sequences underlined | |
| Pseudo intron | 5′ | AUCUUUUUUUUUUUUACUUUUUUUUUUUUUUUGU (SEQ ID No. 2) | 3′ |
| sequence | | | |
| Product after | 5′ | Atto390-uaaaaaaaacgaacacucccaaaaaaaaacaaagcuucguuuuuuuua-Atto495 | 3′ |
| splicing | | (SEQ ID No. 3) | |
| sequence | | | |

TABLE 2
DNA Probe

| | | | |
|---|---|---|---|
| Probe | 5′ | Atto390- | 3′ |
| sequence | | taaaaaaaacgaacactcccaaaaaaaaacaaaggcttcATCTTTTTTTTTTT | |
| | | TACTTTTTTTTTTTTCTTTGTtttttttta-Atto495 (SEQ ID No. 4) | |
| Pseudo intron | 5′ | ATCTTTTTTTTTTTTACTTTTTTTTTTTTTTGT (SEQ ID No. 5) | 3′ |
| sequence | | Key: recognition sequences underlined | |
| Product after | 5′ | Atto390-taaaaaaaacgaacactcccaaaaaaaaacaaagcttcgtttttttta-Atto495 | 3′ |
| splicing | | (SEQ ID No. 6) | |
| sequence | | | |

In a further aspect the invention provides a probe wherein the pseudo intron comprises a 5′ splice site, branch point and 3′ splice site. In a preferred embodiment, the 5′ splice site, branch point and 3′ splice site of the pseudo intron are specific for U2snRNP-dependent splicing. Thus in an aspect of the invention a probe is provided, wherein the 5′ splice site and 3′ splice site comprise a terminal dinucleotide selected from the group comprising: AT-GT, GT-AG, GC-AG and AT-AC for DNA probes and when the probe is an RNA probe the dinucleotide is selected from the group comprising: AU-GU, GU-AG, GC-AG and AU-AC.

In an embodiment a probe is provided, wherein the branch point comprises an adenosine nucleotide.

In an embodiment the probe of the invention has a signal generating component which comprises a Förster (Fluorescence) Resonance Energy Transfer (FRET) reporter group pair or a chemical crosslinking group. The design of the probes is such that when the pseudo intron has yet to be spliced out the reporter pair are kept at distance from each other and are brought into proximity after splicing to generate a detectable signal.

In an aspect of the invention the FRET pair is selected from the group:
a) Europium and a fluorophore, wherein the fluorophore is excited by the Europium emission peak at 620 nm;
b) Terbium and a fluorophore, and the fluorophore is excited by the Terbium emission peak at 495 nm; or
c) Samarium and a fluorophore and the fluorophore is excited by the Samarium emission peak at 350 nm.
d) Containing a FRET pair wherein the signal generating component comprises a fluorescence signal donor located in or attached to the first pseudo exon and the signal acceptor located on the second pseudo exon, kept outside of the proximity required for resonance transfer to take place by the length and presence of the pseudo intron. On splicing of the intron, the FRET pair is positioned with sufficient proximity for FRET to occur. Suitable FRET pairs are well known in the art and may be selected using the FPBase FRET calculator, see: www.fpbase.org/fret/and www.nature.com/articles/s41598-018-35535-9

In an alternative embodiment there is provided a probe wherein the signal generating component comprises a fluorescence signal located in or attached to either the first pseudo exon or the second pseudo exon, and a quencher located in or attached to the pseudo intron. On splicing of the intron, the quencher is removed and thus a detectable signal is generated. Suitable quenchers are well known in the art (Mary Katherine Johansson (2006) Choosing Reporter-Quencher Pairs for Efficient Quenching Through Formation of Intramolecular Dimers; Methods in Molecular Biology⋅February 2006 DOI: 10.1385/1-59745-069-3:17) and are freely available from multiple commercial suppliers.

In a preferred embodiment of the invention there is provided a probe as herein described wherein the probe is designed to form a hairpin loop structure, and wherein the binding motif is positioned predominantly in the loop sequence.

The present invention additionally provides a method for the detection of a functional splicing protein in a sample comprising contacting a probe as herein described with a sample such that in the presence of the functional splicing protein the pseudo intron is excised out of the probe thereby generating a detectable signal to show the presence of the functional splicing protein in the sample.

In an embodiment the splicing protein that is detected in the sample is selected from TDP-43 or FUS or an isoform thereof. In an embodiment the method of the invention is carried out wherein the probe is contacted with the sample in the presence of ATP. When ATP is present it is preferred that the probe is a DNA probe.

The present invention in an embodiment provides the use of a probe described herein for the in vitro detection of a functional splicing protein or mutant forms or misfolded isoforms thereof. Mutant or misfolded isoforms will have less splicing functionality than normal protein.

The present invention further provides a kit comprising a probe as described herein optionally with one or more product sequences as controls, and buffers.

### BRIEF DESCRIPTION OF FIGURES

FIG. 1 is a schematic of splicing of pre-mRNA to mRNA.

FIG. 2 shows a schematic of the invention TDP 43 probe of the invention, which includes a polypyrimidine tract having sequence 5′-YUNAYYYYYYYYYYYYAGG-3′ (SEQ ID NO: 7).

FIG. 3 shows the design of the probe in a hairpin loop

FIG. 4 is a schematic of the processing of the probe of the invention upon splicing

FIG. 5 shows the time dependence of splicing activity in both yeast and human lysates with RNA and DNA probes.

FIG. 6 shows the concentration dependence of the splicing activity in human and yeast lysates

FIG. 7 shows the splicing of the probe of the invention with TDP 43

FIG. 8 shows the amplification of signal when rTDP-43 is added to CSF samples

### DETAILED DESCRIPTION OF THE INVENTION

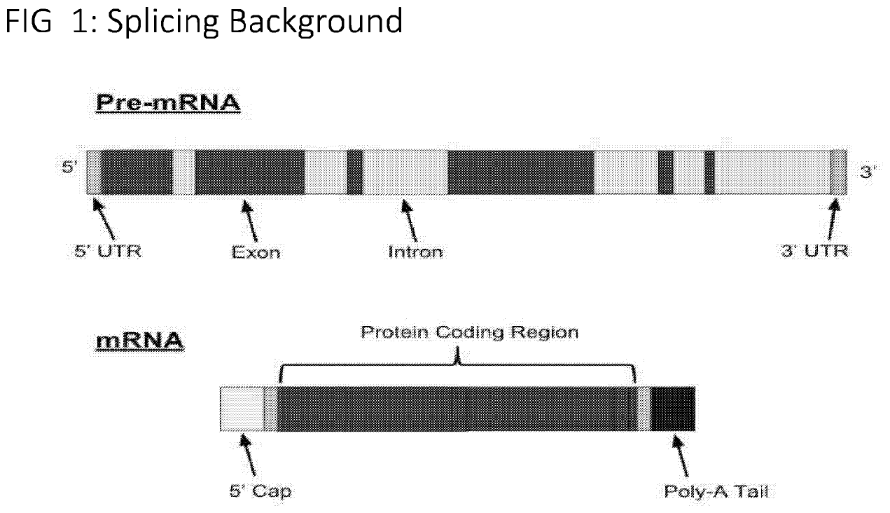

The present invention provides for the first time a method of detecting a splicing protein utilising the proteins functional splicing activity (FIG. 1). In general terms a nucleic acid probe is introduced to a sample and if the particular splicing protein specific for the probe is present, the splicing protein will splice the probe thereby generating a signal.

### Definitions

As used herein the term pseudo exon is used to describe a part of the probe of the present invention that remains after the pseudo intron has been excised due to activity of a functional splicing protein. Two pseudo exons are ligated following excision of a central pseudo intron.

As used herein the term pseudo intron is used to describe a part of the probe that is excised following activity of a functional splicing protein.

As used herein a splice site means a distinct dinucleotide pair located at the 5′ and a second distinct pair at the 3′ end of the pseudo intron and marks the boundary of the probe that will be excised following binding to the functional splicing protein.

As used herein a splicing protein means an RNA and/or DNA binding protein involved in splicing of pre-mRNA to mature messenger RNA (mRNA).

As used herein splicing means the biological process that removes introns (or pseudo intron) from a nucleic acid.

As used herein isoform is a member of a set of proteins with either highly similar or identical amino-acid sequences that originate from a single gene and exhibit isomers due to alternate folding. Protein isoforms may be formed from an mRNA that has undergone alternative splicing mechanisms, or post transcriptional modification of a single gene. Protein isoforms may have different tertiary and quaternary structures, with possible effects on their function and pathogenicity.

As used herein binding means the protein-nucleic acid interaction between a probe of the invention and the splicing protein due to the respective binding motifs, that need to be detected to enable the splicing the probe.

As used herein a homogeneous assay refers to an assay format allowing to make an assay-measurement by a simple one-step mix and read procedure without the necessity to process samples without prior separation or subsequent washing steps.

### Design of Probe

The probe of the invention comprises a nucleic acid sequence that is arranged to have the following elements:
a) A first pseudo exon sequence, a pseudo intron and a second pseudo exon wherein the pseudo intron contains a 5′ splice site, a multiple repeat (e.g. UU/TT or UG/TG rich regions with at least 4 repeat units, a branch point and a 3′ splice site;
b) a further repeat nucleotide motif as a recognition element for binding with a splicing protein; and
c) a signal generating component for generation of a signal upon excision of the pseudo intron and ligation of the first pseudo exon and the second pseudo exon.

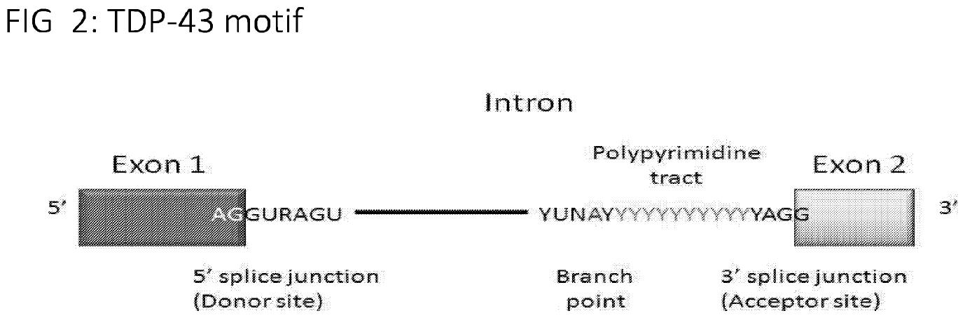

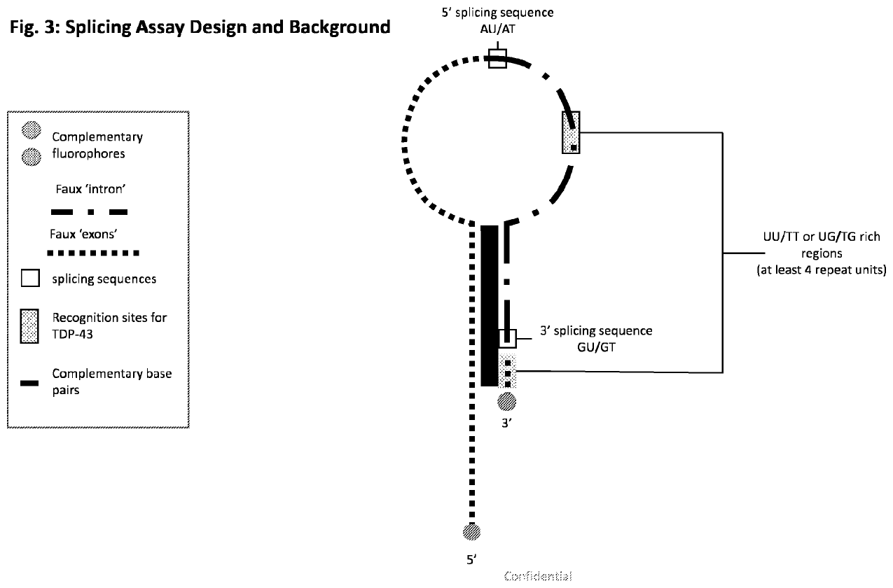

The probe may be DNA or RNA. FIG. 2 demonstrates the general arrangement of the components of the probe for an exemplary TDP 43 probe. FIG. 3 demonstrates the arrangement of the components of the probe when provided as a hair pin loop.

In order for splicing to occur the nucleic acid probe is bound to the target splicing protein via the repeat nucleotide motif. The repeat nucleotide motifs of protein are unique to that protein. For any given splicing the protein there may be more than one sequence that will bind, but those sequence will not bind to different proteins. In a preferred embodiment the highest affinity binding motif for the protein is always chosen. Affinity of the various sequences may be calculated using techniques known in the art.

The probes are between 100 and 50 base pairs. With a minimum of 30% and maximum of 65% of the probe representing the intron. Repeat motifs must appear in a minimum of 35% of the probe, with the repeat motifs repeating unbroken no less than four times.

In an embodiment the repeat nucleotide motif is specific for TDP 43. The two RNA recognition motifs (RRM) of TDP-43 are known to play an active role in binding and alternative splicing of RNA and DNA, with RRM1 posited as the main binding site, assisted by RRM2. UU/UG/GU rich repeats bind with high affinity to RRM1, even when interspersed by other nucleotides. Accordingly, the RNA probes of the invention for TDP 43 comprise UU/UG/GU rich repeats and in a preferred embodiment comprise at 4 to 15 repeats, preferably 4 to 10 repeats, typically about 8 repeats. This enables the association and dissociation constants of the protein to allow for binding to occur, splicing to take place, and diffusion of the spliced sequence.

It will be appreciated that when the probe is a DNA probe the Uracil (U) will be replaced by Thymine (T).

In an embodiment the repeat nucleotide is specific for FUS. Preferred repeats are: GGUG, subsequently CUGG, UGGU, GCUG, CCUG, UGGG and GUGG and UUAGGG. UUAGGG is most preferred. The probe for FUS preferably contains at least 4 to 15 repeats. In an embodiment the repeats above can be mixed. As above, when the probe is a DNA probe the U will be replaced with T.

By adjusting the elements described herein, a person skilled in the art can apply this probe to study a variety of splicing proteins, and their respective isoforms. The online database RBPDB (rbpdb.ccbr.utoronto.ca/index.php) lists a number of RNA binding proteins and their respective functional RRMs, enabling a person skilled in the art to apply the invention disclosed herein to study a variety of splicing proteins. Further, a number of essential sequences that could serve as recognition elements are known in the art (see for example: Ro-Choi, T. S. & Chun Choi, Y. (2012) Chemical Approaches for Structure and Function of RNA in Postgenomic Era. Journal of Nucleic Acids, 369058). The splice sites are a small parts of the pseudo intron located at either end of the intron and with a set base pair in the middle, closer to the 3′ than the 5′ end and typically and preferably contains adenine, but can be cytosine. This set nucleotide is the branch point. The splice site consists of a group of known codes usually between 2-4 bp long and marks the boundary of where the splicing of the intron takes place. Splicing sequences are universal for all mammalian introns and have the following sequences: GT-AG, GC-AG or GT-AG, AT-AC or for RNA GU-AG, GC-AG, or GU-AG, AU-AC.

Typically, the length of the probe is 50-100 base pair long, eg 70, 80, 90 base pairs, with an Intron representing between 30 to 65% of the probe and the repeat motifs must appear in a minimum of 35% of the probe, with motifs repeating unbroken no less than four times.

The recognition sequences in an embodiment make up to 35, 40, 45, 50, 55% of the nucleotides in the probe and may be located within either the pseudo exon or pseudo intron or overlap said intron and exon.

When the probe is RNA it is preferred to modify the RNA to provide resistance to RNA nucleases. Typical modifications comprise alkylating the 2′ OH on the sugar moiety. In a preferred embodiment all the 2′OH moieties are alkylated, and are preferably methylated.

In an embodiment the present invention provides probe that is a hairpin nucleic acid comprising a complimentary 3′ and 5′ stem sequence to form a double stranded stem portion and a single stranded loop portion.

The signal generating component of the probes comprises one or more groups capable of generating a signal through fluorescence. In one embodiment the signal generating component of the Loop probes comprises a Förster (Fluorescence) Resonance Energy Transfer (FRET) reporter group pair of donor and acceptor fluorophores such that in the presence of the pseudo intron the acceptor and donor fluorophore are separated to prevent resonance transfer and generation of a detectable. On excision of the Intron the pairs are brought together to generate a detectable fluorescent signal. In an embodiment the reporter pair generates a detectable signal the donor and acceptor fluorophores close enough to enable Förster (Fluorescence) Resonance Energy Transfer (FRET) or for a pair or a chemical crosslinking group(s) to form a chemical cross-link. In an alternative embodiment, the probes carry biotin moieties as binding points for biotin-streptavidin detection systems.

Preferably, the fluorophore in the FRET pair is one that is excited by one of the emission peaks emitted by a Lanthanide. More preferably, the first fluorophore has DOTA (dodecane tetra acetic acid) complexed with Europium (Eu) bound to the base of the first 5′ nucleotide and the other pair is bound to the base of the final 3′ nucleotide.

In a further embodiment, a FRET pair may be chosen wherein the signal generating component comprises a fluorescence signal donor located in or attached to the first pseudo exon and the signal acceptor located on the second pseudo exon, kept outside of the proximity required for resonance transfer to take place by the length and presence of the pseudo intron. On splicing of the intron, the FRET pair is positioned with sufficient proximity for FRET to occur. Suitable FRET pairs are well known in the art and may be selected using the FPBase FRET calculator, see: www.fpbase.org/fret/ and www.nature.com/articles/s41598-018-35535-9

In an alternative embodiment the pseudo intron is labelled with a quencher to prevent the generation of a detectable signal and once the intron is excised the signal is detectable. Such quenchers are known in the art Mary Katherine Johansson (2006) Choosing Reporter-Quencher Pairs for Efficient Quenching Through Formation of Intramolecular Dimers; Methods in Molecular Biology·February 2006 DOI: 10.1385/1-59745-069-3:17) and are freely available from multiple commercial su

Depending on the signalling method used, appropriate readers are commercially available for detection.

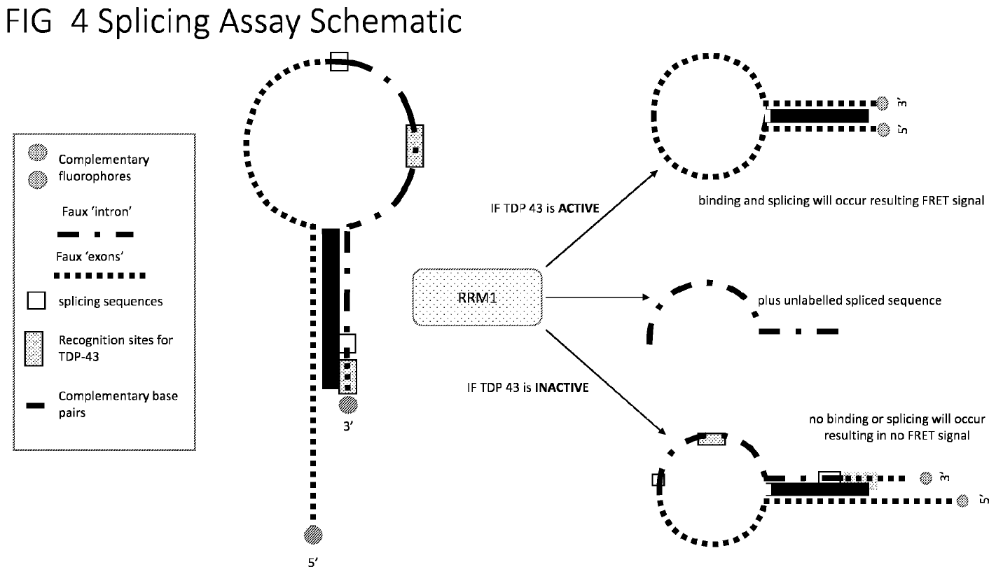

The probes of the invention, are in preferred embodiments, provided as hair pin loops. In such embodiments, the probe comprises a 5′ stem sequence, a target loop, a 3′ stem sequence and a signal generating component. The stem sequence hybridise to each other to form a double stranded portion and leaving a single stranded loop structure. On splicing the intron is excised and in an embodiment the product of the splicing also forms a smaller hair pin loop which allow for the FRET pairs to come into close proximity (see FIG. 4).

Each probe has a unique RNA recognition motif sequence(s) for the target splicing protein. Those motifs can be determined by or are known in the art. It is possible to target a large range of splicing proteins, by simple adaptation of the existing probes. Using online databases such as the RBPDB (rbpdb.ccbr.utoronto.ca/index.php) and the existing literature known splicing proteins, details of their target RRMs and their binding motifs can be incorporated into the probe design of the invention and the recognition sequences incorporated. When new splicing proteins are identified, it will be possible to use the natural sequences that are spliced by the protein in question and these can be, aligned and the homology of these sequences with the reference to the expected tandem repeats identify the binding sites of said protein.

Splicing proteins have different isoforms and often this results in different folding patterns making the RRM more or less accessible to a splicing probe and can result in reduced splicing efficiency. The folding differences can be exploited by altering the shape of the probe when it interacts with the splicing protein. Modifying the probe at the beginning of the loop structure with one or more pseudo uridines preferably via the replacement of two three or four uridine permits different confirmation of probes.

### Sample Preparation

Any biological sample can be used in conjunction with the probe and assay of the invention. Where the sample is a solid tissue the sample is homogenised and prepared in a suitable buffer. For blood, cerebral spinal fluid, sputum or other aqueous solutions the probe is added to the sample and the signal is detected by methods known in the art. Assay design is such that liquid samples such as blood or CSF can be added directly to microtiter plates in the volumes stated. In the case of solid samples or exosome extraction standard homogenisation and ultracentrifugation methods are employed (Goldberg (2008) Mechanical/physical methods of cell disruption and tissue homogenization. Methods Mol Bio. 424: 3-22).

Using a standard curve with known concentrations of a splicing protein, it is possible to quantify the respective concentration of the target splicing protein in an unknown sample. Furthermore, using substrates with alternative configurations induced by the insertion of modified bases into the binding motif may enable the differentiation between a healthy and pathological splicing apparatus. Each specific probe may carry a specific label for each isoform, and enable the detection of the presence of such isoform. Standards then can be established to determine the level of normal splicing activities and assays can be run to determine whether samples contain abnormal level of splicing and from this infer pathological status of the sample.

Thus, the present invention describes a probe that can be used for the detection of a functional splicing protein. The invention now will be exemplified by the following non-limiting examples.

### Examples

All experiments were conducted in triplicate on 384 plates with protective film covers. Mean averages were calculated, background signal of buffer and the relevant probe was then deducted to achieve final number of counts declared on figures.

### Cell Acquisition

Humans and yeast cells were collected via centrifugation at 3500 g for 5 minutes. Lysates were then acquired via standard ultracentrifugation for Human cells (see for example: Goldberg (2008) Mechanical/physical methods of cell disruption and tissue homogenization. Methods Mol Bio. 424: 3-22) and standard glass bead beating for Yeast cells (see for example: DeCaprio and Kohl (2020) Lysing Yeast Cells with Glass Beads for Immunoprecipitation. Cold. Spring Harb Protoc. 5. doi:10.1101/pdb.prot098590). Total protein concentration was measured and the concentration of the two was normalised to 0.5 mg/ml.

### Nucleic Acid Probes

The RNA probe (SEQ ID No. 1) and DNA probe (SEQ ID No. 4) used throughout experimentation were chemically synthesised, the full sequences for these can be found in Table 1 and 2. Controls included, these probes on their own, the pseudo intron (SEQ ID No. 2 and 5) and the product after splicing (SEQ ID No. 4 and 6) again detailed in full on Tables 1 and 2.

The nucleic acid probes arrived lyophilised and are reconstituted in RNA free water to a concentration of 100 pmol, this stock is then aliquoted and stored at −20° C. for long term storage and 4° C. for short term storage. ATP (Sigma Aldrich) was too reconstituted with RNA free water, to a concentration of (1 mg/ml).

Human CSF used was a pooled sample group and the recombinant TDP-43 was produced from human cell line. Assay design is such that liquid samples such as blood or CSF can be added directly to microtiter plates in the volumes stated. In the case of solid samples or exosome extraction standard homogenisation and ultracentrifugation methods are employed (Goldberg (2008) Mechanical/physical methods of cell disruption and tissue homogenization. Methods Mol Bio. 424: 3-22).

### Time Dependency of Splicing Activity Using Lysates

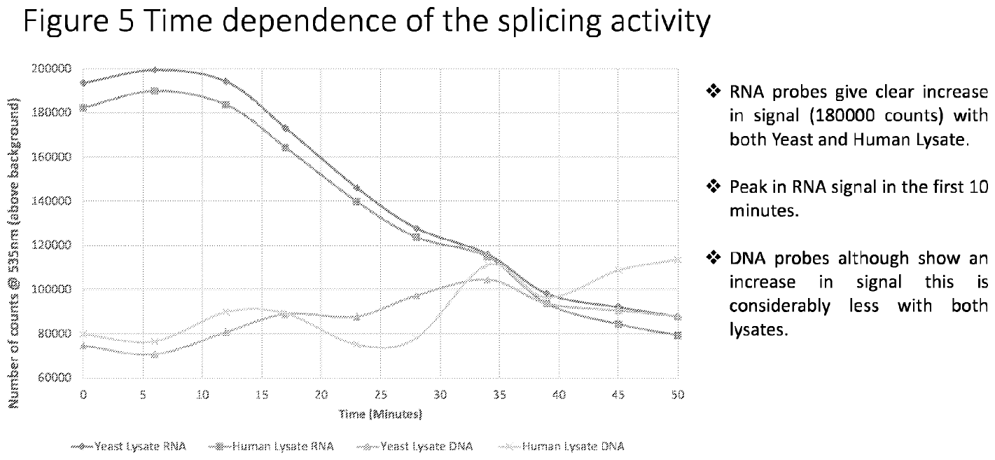

Probe (DNA or RNA) (1 μl containing 100 pmol) and 2 μl of lysate (Human or Yeast) was added to artificial cerebral spinal fluid (ACSF, NaCl 125 mM, NaHCO₃ 26 mM, NaH₂PO₃ 1.25 mM, KCl 2.5 mM) to a total volume of 100 μl. This was incubated at 37° C. with orbital shaking, with 535 nm counts measured periodically. FIG. 5 shows the time dependence of the splicing activity of both DNA and RNA probes (SEQ ID No. 4 and SEQ ID No. 1), revealing a peak in RNA splicing activity early on in the experiment (between 0 and 10 minutes). DNA splicing activity in the context of just the lysates however appears to slowly increase over the duration of the experiment, until approximately 35 minutes. Providing evidence for DNA/RNA binding within our samples and demonstrating that our probes are undergoing the predicted cellular modifications ‘being spliced’ in order to produce a signal.

### Concentration Dependency of Splicing Activity Using Lysates

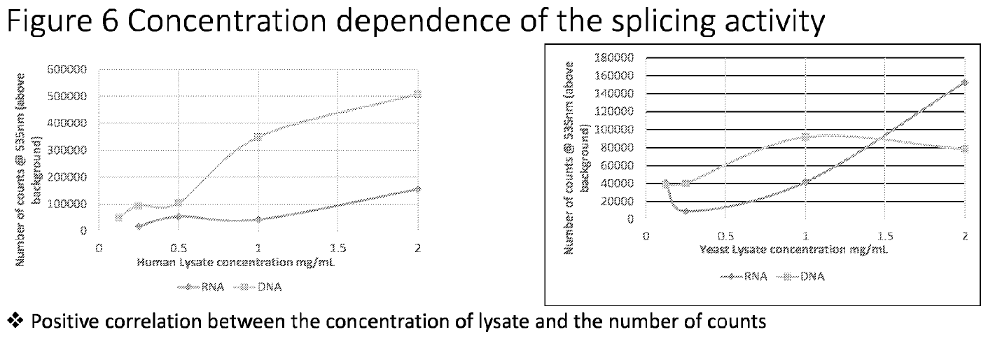

Probe (DNA or RNA) (1 μl containing 100 pmol) and varying amounts (from 0.25 mg/ml to 2 mg/ml) of lysate (Human or Yeast) was added to artificial cerebral spinal fluid (ACSF, NaCl 125 mM, NaHCO₃ 26 mM, NaH₂PO₃ 1.25 mM, KCl 2.5 mM) to a total volume of 100 μl. This was incubated at 37° C. with orbital shaking for 10 minutes at which time 535 nm counts measured on a Perkins Elmer plate reader. FIG. 6 shows the concentration dependence of the splicing activity of both DNA and RNA probes (SEQ ID No. 4 and SEQ ID No. 1) using lysates. As the concentrations of the cellular lysates increases so does the incumbent concentration of TDP-43 to act on the probes, splicing them, and therefore signal increases.

### Splicing Activity of Recombinant TDP-43 Using DNA Probes

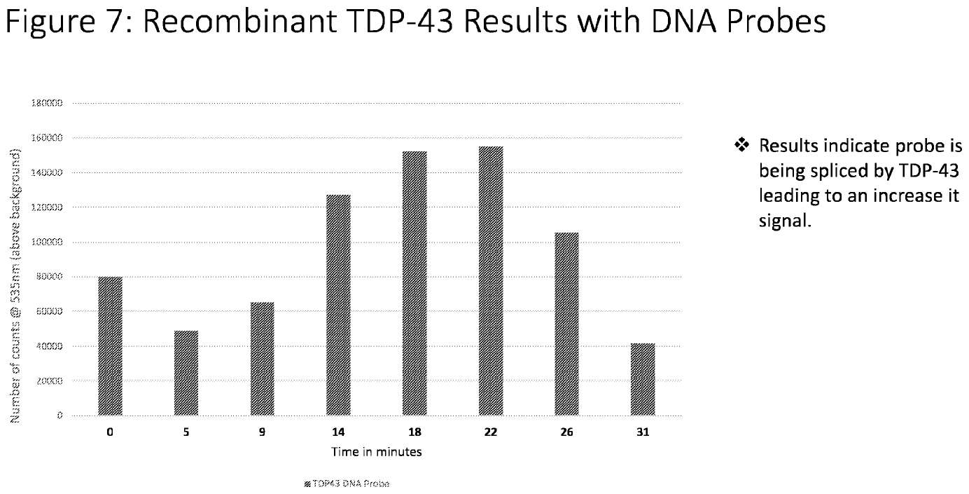

DNA probe (1 μl containing 100 pmol), 1 μl of Recombinant TDP-43 and 1 μl of ATP (1 mg/mL) was added to artificial cerebral spinal fluid (ACSF, NaCl 125 mM, NaHCO₃26 mM, NaH₂PO₃ 1.25 mM, KCl 2.5 mM) to a total volume of 100 μl. This was incubated at 37° C. with orbital shaking for 10 minutes at which time 535 nm counts measured with 535 nm counts measured periodically, from 0 minutes to 31 minutes. FIG. 7 shows the increase in signal from DNA probes (SEQ ID No. 4) with only TDP-43 present. This provides clear evidence that TDP-43 is the protein responsible for acting on the probes. A peak in activity is seen at approximately 20 minutes, following this the signal is lost likely due to cellular degradation. It is noteworthy that this experiment was attempted without the addition of ATP and produced no signal, leading to the conclusion that this process is ATP dependant as would be expected of splicing activity.

### Splicing Activity of Human CSF and Recombinant TDP-43 Using DNA Probes, Independently and Collectively

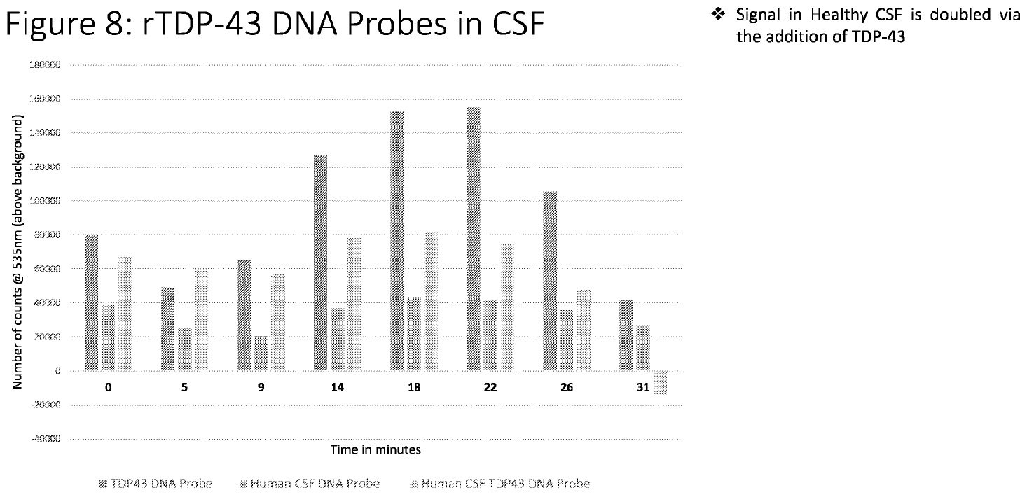

DNA probe (1 μl containing 100 pmol), 1 μl of Recombinant TDP-43 and 1 μl of ATP (1 mg/mL) was added to artificial cerebral spinal fluid (ACSF, NaCl 125 mM, NaHCO₃26 mM, NaH₂PO₃ 1.25 mM, KCl 2.5 mM) to a total volume of 100 μl. Simultaneously, DNA probe (1 μl containing 100 pmol) was added to Human cerebral spinal fluid (both with and without the addition of the recombinant TDP-43) to a total volume of 100 μl. This was incubated at 37° C. with orbital shaking for 10 minutes at which time 535 nm counts measured with 535 nm counts measured periodically, from 0 minutes to 31 minutes. FIG. 8 shows the recombinant data from FIG. 7 alongside the increase in signal from DNA probes (SEQ ID No. 4) in the human CSF with and without the addition of TDP43. Here we see a similar trend in peak signal for all groups, with peak activity at approximately 20 minutes. Although Human CSF alone has a relatively lower signal, this would be expected given that cellular concentrations of TDP-43 in the cell line used are known to be very low. When rTDP-43 is added to the Human CSF (additive to its native concentration) a predicted signal increase is seen. However, this signal increase is not as high as when the rTDP-43 acts alone, again this is correlates well with current understanding as TDP-43 is responsible for many cellular roles is it likely that any additional TDP-43 would not be entirely ‘focused’ on the role of splicing, whereas in samples where the only actionable role for rTDP-43 is the available probe, a ‘focused’ or sole action of TDP-43 is being carried out and so signal would therefore higher.

1. A nucleic acid probe for use in the detection of a functional splicing protein, the probe comprising:
a) a pseudo intron flanked by a first pseudo exon and a second pseudo exon; wherein the pseudo intron contains a 5′ splice site, a branch point and a 3′ splice site;
b) a recognition element for binding with a splicing protein; and
c) a signal generating component for generation of a signal upon excision of the pseudo intron and ligation of the first pseudo exon and the second pseudo exon.

2. The nucleic acid probe of claim 1, wherein the probe is a DNA probe.

3. The nucleic acid probe of claim 1, wherein the probe is an RNA probe.

4. The nucleic acid probe of claim 3, wherein a 2′OH of the RNA sugar moiety of an RNA nucleotide of the RNA probe is alkylated.

5. The nucleic acid probe of claim 4, wherein the 2′OH of the RNA sugar moiety of the RNA nucleotide is methylated.

6. The nucleic acid probe of claim 1, wherein the recognition element comprises a first recognition sequence and a second recognition sequence for interaction with a splicing protein.

7. The nucleic acid probe of claim 1, wherein the first recognition sequence is located in the pseudo intron, and the second recognition sequence is located in either the first pseudo exon or the second pseudo exon.

8. The nucleic acid probe of claim 1 wherein the recognition sequences bind to a splicing protein selected from the group of heterogeneous nuclear ribonucleoproteins (hnRNPs).

9. The nucleic acid probe of claim 1 wherein the recognition sequences bind to the splicing protein TAR DNA-binding protein 43 (TDP-43) or Fused in Sarcoma (FUS).

10. The nucleic acid probe of claim 9, wherein at least one of the recognition sequences binds to the RRM1 domain of TDP-43.

11. The nucleic acid probe of claim 1, wherein the 5′ splice site, branch point and 3′ splice site of the pseudo intron are specific for U2snRNP-dependent splicing.

12. The nucleic acid probe of claim 1, wherein the 5′ splice site and 3′ splice site comprise a terminal dinucleotide selected from: GT-AG, GC-AG or GT-AG, AT-AC when the probe is a DNA probe and when the probe is a RNA probe the terminal dinucleotide is selected from GU-AG, GC-AG, or GU-AG, AU-AC.

13. The nucleic acid probe of claim 1, wherein the branch point comprises an adenosine nucleotide.

14. The nucleic acid probe of claim 1, wherein the signal generating component comprises a Förster (Fluorescence) Resonance Energy Transfer (FRET) reporter group pair or a chemical crosslinking group.

15. The nucleic acid probe of claim 15, wherein the FRET pair is selected from the group:
a) Europium and a fluorophore, wherein the fluorophore is excited by the Europium emission peak at 620 nm;
b) Terbium and a fluorophore, and the fluorophore is excited by the Terbium emission peak at 495 nm; or
c) Samarium and a fluorophore and the fluorophore is excited by the Samarium emission peak at 350 nm.

16. The nucleic acid probe of claim 1, wherein;
the signal generating component comprises a fluorescence signal located in or attached to either the first pseudo exon or the second pseudo exon, and a quencher located in or attached to the pseudo intron; and/or
the nucleic acid probe has a hairpin loop structure.

17. (canceled)

18. A method for the detection of a functional splicing protein in a sample, the method comprising contacting the nucleic acid probe of claim 1 with a sample such that in the presence of the functional splicing protein, the pseudo intron is excised out of the probe, thereby generating a signal that is detected to show the presence of the functional splicing protein in the sample.

19. The method of claim 18, wherein;
the splicing protein is TDP-43 or FUS; and/or
the nucleic acid probe is contacted with the sample in the presence of ATP.

20. (canceled)

21. Use of the nucleic acid probe of claim 1 for the in vitro detection of a functional splicing protein or mutant forms or misfolded isoforms thereof.

22. A kit comprising the nucleic acid probe of claim 1.


SEQUENCE LISTING (7 sequences)

SEQ ID NO: 1 - RNA, 83 nt, Artificial
  Synthetic
uaaaaaaaacgaacacucccaaaaaaaaacaaaggcuucaucuuuuuuuuuuuuacuuuuuuuuuuuucuuuguuuuuuuuua

SEQ ID NO: 2 - RNA, 34 nt, Artificial
  Synthetic
aucuuuuuuuuuuuuacuuuuuuuuuuuuuuugu

SEQ ID NO: 3 - RNA, 48 nt, Artificial
  Synthetic
uaaaaaaaacgaacacucccaaaaaaaaacaaagcuucguuuuuuuua

SEQ ID NO: 4 - DNA, 83 bp, Artificial
  Synthetic
taaaaaaaacgaacactcccaaaaaaaaacaaaggcttcatcttttttttttttacttttttttttttctttgttttttttta

SEQ ID NO: 5 - DNA, 34 bp, Artificial
  Synthetic
atcttttttttttttactttttttttttttttgt

SEQ ID NO: 6 - DNA, 48 bp, Artificial
  Synthetic
taaaaaaaacgaacactcccaaaaaaaaacaaagcttcgtttttttta

SEQ ID NO: 7 - RNA, 19 nt, Artificial
  Synthetic
  misc_feature (3)..(3): n is a, c, g, or u
yunayyyyyyyyyyyyagg